(19) United States
(12) Patent Application Publication
(10) Pub. No.: US 2006/0241163 A1
(43) Pub. Date: Oct. 26, 2006

(54) Optically active (S)-hydantoin derivative

(76) Inventors: Kaoru Okamoto, Hyogo (JP); Akira Ishii, Hyogo (JP); Nobuhisa Okukado, Hyogo (JP); Hirohide Matsuura, Hyogo (JP); Fujio Naka, Hyogo (JP)

Correspondence Address: HOLLANDER LAW FIRM, P.L.C., SUITE 305, 10300 EATON PLACE, FAIRFAX, VA, 22030, US

(21) Appl. No.: 11/110,267

(22) Filed: Apr. 20, 2005

(51) Int. Cl. A61K 31/4166

(52) U.S. Cl. 514/389; 548/317.5

(57) ABSTRACT

(S)-(−)-5-hydroxy-1-methylhydantoin is useful as a drug having high safety. Since the compound of the present invention, (S)-(−)-5-hydroxy-1-methylhydantoin, rarely changes to the metabolite having high toxicity compared with the R-form and the racemate, it is preferred as an agent for renal failure especially when used for chronic renal insufficiency in a patient where toxic substances are not excreted, but are accumulated in the body due to disturbance of renal functions.

### FIELD OF THE INVENTION

The present invention relates to an optically active substance of 5-hydroxy-1-methylimidazolidine-2,4-dione which is useful as a drug.

### BACKGROUND OF THE INVENTION

It has been known that 5-hydroxy-1-methylimidazolidine-2,4-dione (hereinafter referred to as 5-hydroxy-1-methylhydantoin) is useful as a plant growth regulator (Japanese Patent Laid-Open No. Sho-57-114578); a hypoglycemic agent; a diuretic; a hypolipemic agent (U.S. Pat. No. 4,647,574); an improving agent for renal function (U.S. Pat. No. 5,084,473); an eliminating agent for active oxygen and free radical (U.S. Pat. No. 6,197,806); a therapeutic agent for intractable vasculitis (U.S. Pat. No. 6,251,929); and an agent for hypoalbuminemia (U.S. Pat. No. 6,451,831).

In 5-hydroxy-1-methylhydantoin, the 5-position of hydantoin ring is an asymmetric carbon and, therefore, there are optical isomers. However, separation of optical isomers of 5-hydroxy-1-methylhydantoin is difficult and, in an article by Ienaga et al. (J. Chem. Soc. Perkin Trans., I, 1989, 1153-1156), N-benzyloxycarbonylproline derivative was condensed with (±)-5-hydroxy-1-methylhydantoin to give diastereomers. Each diastereomer was separated, but it was not possible that they could be hydrolyzed without racemization to give each optically active isomer (page 1154 of Ienaga et al.). In addition, in the above-mentioned patent publications, there is no description at all about the isolated optical isomers of 5-hydroxy-1-methylhydantoin, about their properties and characteristics, or about the process of separation and production of the optically active substances.

Regarding a compound having an asymmetric center, there are some cases where pharmacological action and toxicity are different between optical isomers. In the case of drugs, it is preferred to utilize an optically active substance having excellent pharmacological action, good pharmacokinetics, or having high safety. Accordingly, with regard to 5-hydroxy-1-methylhydantoin, it also has been demanded that optically active substances thereof be separated and synthesized, and that characteristics of both optically active substances be made clear.

The present inventors have succeeded in separation and synthesis of optically active substances of 5-hydroxy-1-methylhydantoin and have found that the S-form has particularly low conversion rate to toxic metabolites and high safety in view of toxicity. An object of the present invention is to provide (S)-(−)-5-hydroxy-1-methylhydantoin which is useful as a drug for renal failure.

### SUMMARY OF THE INVENTION

The present invention relates to (S)-(−)-5-hydroxy-1-methylhydantoin which is useful as a drug having a high safety. With regard to optically active substances of 5-hydroxy-1-methylhydantoin, there are (S)-(−)-5-hydroxy-1-methylhydantoin (S-form) and (R)-(+)-5-hydroxy-1-methylhydantoin (R-form). These two enantiomers are represented by the following structural formulae:

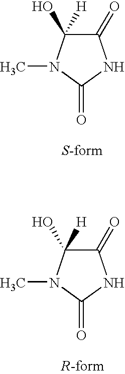

### BRIEF DESCRIPTION OF THE DRAWINGS

FIG. 1 shows results pertaining to pharmacokinetics in blood, when each of (S)-(−)-5-hydroxy-1-methylhydantoin and (R)-(+)-5-hydroxy-1-methylhydantoin was orally administered to model rats for renal insufficiency.

FIG. 2 shows results pertaining to pharmacokinetics in blood, when each of (S)-(−)-5-hydroxy-1-methylhydantoin and the racemate was orally administered to model rats for renal insufficiency.

### DETAILED DESCRIPTION OF THE INVENTION

(S)-(−)-5-Hydroxy-1-methylhydantoin according to the present invention and (R)-(+)-5-hydroxy-1-methylhydantoin, which is an antipode thereof, are able to be produced by: (1) introducing an optically active substituent into (±)-5-hydroxy-1-methylhydaintoin to synthesize and separate diastereomers, and then (2) removing the substituent from each diastereomer. Due to its structure, 5-hydroxy-1-methylhydantoin is racemized or is isomerized to 5-hydroxy-3-methylhydantoin which is a regioisomer for a hydroxyl group under a strongly acidic or strongly basic condition. Therefore, it is necessary to conduct a method of racemate resolution using a condition that is not strongly acidic or strongly basic, for example, a neutral condition. As a substituent suitable in the present process, 1-phenylethoxy group may be exemplified. Thus, for example, (±)-5-hydroxy-1-methylhydantoin is made to react with (S)- or (R)-1-phenylethanol in the presence of an acidic catalyst whereupon diastereomers of 1-methyl-5-(1-phenylethoxy)hydantoin are synthesized. Those diastereomers are able to be separated by silica gel column chromatography. When the separated diastereomers are subjected to a catalytic reduction in the presence of a catalyst under a neutral condition in ethyl acetate and the like, it is possible to convert the diastereomers into each of optically active substances of (S)-(−)- and (R)-(+)-5-hydroxy-1-methylhydantoin.

Alternatively, diastereomers of 1-methyl-5-(1-phenylethoxy)hydantoin are able to be efficiently produced starting from 1-methylhydantoin. Thus, 1-methylhydantoin is treated with bromine in 1,2-dichloroethane and recrystallized from 1,2-dichloroethane to give an iminium salt. The iminium salt is made to react with (S)- or (R)-1-phenylethanol whereupon diastereomers of 1-methyl-5-(1-phenylethoxy)hydantoin are able to be synthesized. In this reaction, it is preferred to remove hydrogen bromide which is produced during the reaction. Hydrogen bromide is able to be removed when a base such as potassium carbonate is made to coexist therewith. However, since there is a possibility that position isomerism for a hydroxyl group takes place under a basic condition, a method of removing hydrogen bromide using an acid scavenger such as molecular sieve 4A is preferred. When molecular sieve 4A is suspended in 1,2-dichloroethane and mixed with (R)- or (S)-1-phenylethanol and 1 to 2 equivalent(s) of 1-methylhydantoin iminium salt at 35 to 45° C., diastereomers 2a and 2b are produced from (R)-1-phenylethanol in a ratio of 1:1 or diastereomers 3a and 3b are produced from (S)-1-phenylethanol in a 1:1 ratio:

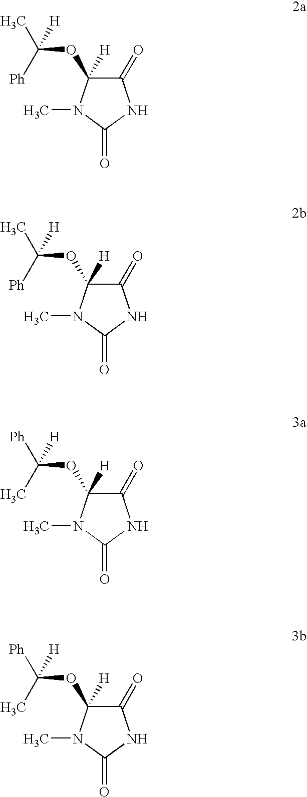

The above diastereomers are separable by column chromatography and may, for example, be separated by at least one of flash chromatography using silica gel, medium-pressure liquid chromatography, or chiral chromatography. Other resolution methods may also be employed, such as recrystallization. Each separated diastereomer is subjected to a catalytic reduction in the presence of a catalyst, and (S)-(−)-5-hydroxy-1-methylhydantoin and (R)-(+)-5-hydroxy-1-methylhydantoin are able to be produced in a good yield from 2a and 3b and from 2b and 3a, respectively.

An optically active substance of 5-hydroxy-1-methylhydantoin may also be obtained by direct optical resolution of (±)-5-hydroxy-1-methylhydantoin by a high-performance liquid chromatography (HPLC) using an appropriate chiral column. For example, it is possible to conduct a direct optical resolution of a racemate of 5-hydroxy-1-methylhydantoin using a chiral column (CHIRALPAK AS; Daicel). The column is filled with a stationary phase bed of silica gel coated with amylose where (S)-1-phenylethyl carbamate is chemically bonded thereto. This HPLC method is also able to be used for assay of optical purity of (S)-(−)-5-hydroxy-1-methylhydantoin of the present invention and (R)-(+)-5-hydroxy-1-methylhydantoin which is an antipode thereof.

(S)-(−)-5-hydroxy-1-methylhydantoin of the present invention includes pharmaceutically acceptable salts with an alkali metal such as sodium or potassium; with an alkaline-earth metal such as calcium, magnesium or barium; or with another metal such as aluminium or zinc. These salts may be manufactured by known methods from (S)-(−)-5-hydroxy-1-methylhydantoin of the present invention in a free state or may be mutually converted among the salts. When there are hydrates and complexes of (S)-(−)-5-hydroxy-1-methylhydantoin of the present invention, the present invention includes any of such hydrates and complexes. According to embodiments of the present invention (S)-(−)-5-hydroxy-1-methylhydantoin or a pharmaceutically acceptable salt thereof is optically pure or substantially optically pure, for example, having less than about 2.5% by weight of (R)-(+)-5-hydroxy-1-methylhydantoin.

(S)-(−)-5-hydroxy-1-methylhydantoin of the present invention can be made into pharmaceutical preparations by a combination with one or more suitable pharmaceutical carriers or diluents according to any conventional methods, for example, preparations for oral administrations (e.g. tablets, capsules, powders, liquids) or preparations for parenteral administrations (e.g. for subcutaneous, intravenous, intramuscular, intrarectal and intranasal administrations). The compound of the present invention may also be used in the form of the pharmaceutically acceptable salt, and can be used either solely or jointly together in pharmaceutically effective amounts with pharmaceutically effective amounts of other pharmaceutically effective ingredients for treating animals or humans.

In the case of preparations for oral administration, the compound of the present invention alone, or together with a commonly-used excipient such as one or more suitable additives (e.g. lactose, mannitol, corn starch, potato starch, potassium citrate), may be mixed with a binder such as cellulose derivatives (e.g. crystalline cellulose, hydroxypropylcellulose), gum arabicum, corn starch, or gelatin; a disintegrating agent such as corn starch, potato starch or calcium carboxymethylcellulose; a lubricant such as talc or magnesium stearate; and other agents such as bulking agents, moisturizing agents, buffers, preservatives, perfumes and the like to give tablets, powders, granules or capsules.

In the case of injections, it is possible to prepare the solution, suspension or emulsion in an aqueous solvent such as distilled water for injection; in physiological saline solution and glucose solution for injection; or in a non-aqueous solvent such as plant oil, synthetic fatty acid glycerides, higher fatty acid esters, or propylene glycol. If necessary, conventional excipients such as solubilizing agents, isotonizing agents, suspending agents, emulsifiers, stabilizers and preservatives may be added.

Furthermore, depending upon the type of the disease and patient, it is possible to prepare other preparations than those which were mentioned already, for example, suitable preparations for the therapy, such as syrups, suppositories, inhalations, aerosols, collyriums, medicines for external use (e.g. ointments, gels, poultices) and the like.

The resolved, separated, purified, or isolated optically active (S)-(−)-5-hydroxy-1-methylhydantoin of the present invention may be administered to patients in pharmaceutically effective amounts for treating humans or animals, such as mammals, in need of treatment or prevention of renal failure. The preferred dose of the compound of the present invention may vary depending upon the patient to be administered, the preparation form, the method and term of the administration, and the like. In order to achieve a desired effect, 20-6,000 mg per day, preferably 50-4,000 mg per day, may be usually given to common adults by oral route. In the case of parenteral administration such as by injection, the effect may be expected by a smaller dose than that of oral administration.

Preferred embodiments of the present invention are given as follows:

(1) (S)-(−)-5-hydroxy-1-methylhydantoin and a pharmaceutically acceptable salt thereof.

(2) An agent for renal failure containing the compound according to the above (1) or a pharmaceutically acceptable salt thereof as an effective ingredient.

(3) An agent for renal failure according to the above (2), wherein the agent is a therapeutic or preventive agent for renal failure.

(4) An agent for renal failure according to the above (3), wherein the agent is a therapeutic or preventive agent for acute renal failure.

(5) An agent for renal failure according to the above (3), wherein the agent is a therapeutic or preventive agent for chronic renal failure.

(6) An agent for renal failure according to the above (2), wherein the agent is a suppressive agent for the progression of renal failure.

(7) An agent for renal failure according to the above (2), wherein the agent is a preparation for oral administration.

(8) An agent for chronic renal failure according to the above (2), wherein the agent is a tablet.

The following examples are illustrative only and are not intended to limit the scope of the invention. All parts, percentages and ratios are by weight, all temperatures are in ° C., and all reactions are conducted at about atmospheric pressure and room temperature unless indicated to the contrary:

### EXAMPLES

In the following Examples, an example of a process for producing the compound of the present invention will be illustrated in more detail. Incidentally, in the Examples, the following instruments and reagents were used for the measurement of properties of the substances.

Melting point was measured by a melting point measuring device of type MP-21 (Yamato) and no correction was conducted. Rotary power ([a]_{D}) was measured by a polarimeter of DIP-140 type (Nippon Bunko) using a cell of 10 cm. ¹H-NMR was measured by a nuclear magnetic resonance spectrometer of AM-400 Wb type (Bruker) and tetramethylsilane (TMS) was used as an internal stadard. Analysis of enantiomer by ¹H-NMR was conducted by addition of a shift reagent [Eu(tfc)₃, 30 mg] to a heavy acetone (6 mL) solution containing 5-hydroxy-1-methylhydantoin (5 mg). Infrared spectrum (IR) was measured by a KBr method using a Fourier transform infrared spectrophotometer of FT-200 type (Horiba). Elementary analysis was conducted using an elementary analysis apparatus of a CHN Corder MT-5 type (Yanako).

With regard to thin-layer chromatography (TLC), a silica gel plate (Art. 5715) manufactured by Merck was used, development for 5 cm was conducted using a solvent of ethyl acetate/hexane (1:1) and detection was done for ultraviolet absorption wavelength of 254 nm. With regard to silica gel column chromatography, silica gel 60 (Merck, Art. 7734) was used and elution was conducted with benzene/ethyl acetate (1:1). With regard to flash chromatography, silica gel 60 (Merck, Art. 9385, 50 mm φ×180 mm) was used and elution was conducted using a hexane/ethyl acetate (3:2) solution. In medium-pressure liquid chromatography, a column filled with spherical silica gel CQ-3 (Wako, 45 mm φ×500 mm) was used. A hexane/ethyl acetate (2:1) solution was used for a mobile phase and elution was conducted at a flow rate of 20 mL/minute using a low-prep pump of 81-M-2 type manufactured by Chemco. The eluate was subjected to detection by ultraviolet absorption wavelength of 254 nm using an ultraviolet detector of Uvicon TV-750L type (manufactured by Toyo Kagaku Sangyo).

Analysis by HPLC was conducted by a chromatographic system comprising a pump of LC-6A type (Shimadzu), an automatic sampler of AS-L350 type (Nippon Bunko), an ultraviolet detector of Uvidec-100-VI type (Nippon Bunko) and a data processing apparatus of DS-L 300 type (Nippon Bunko) using Chiralpak AS (4.6 mm φ×250 mm, Daicel) as a column. Column temperature was made 0° C., a hexane/ethanol/acetic acid (85:14.9:0.1, v/v) was used as a mobile phase, elution was conducted at a flow rate of 1.0 mL/minute and detection was done by an ultraviolet absorption wavelength of 215 nm.

With regard to (R)-1-phenylethanol (>99% e.e.) and (S)-1-phenylethanol (>97% e.e.), those manufactured by Chisso Corporation were used. Absolute configurations of position 5 of the optically resolved (+)- and (−)-5-hydroxy-1-methylhydantoin were confirmed to be R-form for a (+)-substance and S-form for a (−)-substance by a method mentioned in Ienaga et al. (J. Chem. Soc. Perkin Trans. I, 1989, 1153-1156).

### EXAMPLE 1

A mixture of 106.8 g (0.82 mol) of (±)-5-hydroxy-1-methylhydantoin, 25.0 g (0.21 mol) of (S)-1-phenylethanol and 1.0 g of p-toluenesulfonic acid was suspended in 2.5 L of benzene in a flask equipped with a Dean-Stark's water separator and heated to reflux for 48 hours until no more water was absolutely resulted therefrom. After the insoluble matters were filtered off, a benzene solution was washed with a diluted aqueous solution of sodium hydrogen carbonate. After drying over anhydrous sodium sulfate, it was concentrated in vacuo to give 40.0 g of a 2.5:1 mixture of two diastereomers, namely (5R,1′S)-1-methyl-5-(1′-phenylethoxy)hydantoin (3a) and (5S,1′S)-1-methyl-5-(1′-phenylethoxy)hydantoin (3b), as a colorless oily substance. The mixture was separated by column chromatography using 6 L of silica gel where benzene/ethyl acetate (1:1) was an eluate to give crude crystals of the diastereomer 3a (17.0 g) and 3b (4.0 g). Each of the crude crystals was recrystallized from ethyl acetate/petroleum ether to give 15.5 g (32%) of the diastereomer 3a and 2.5 g (5%) of the diastereomer 3b as white crystals.

The same treatment was carried out using 106.8 g (0.82 mol) of (±)-5-hydroxy-1-methylhydantoin and 25.0 g (0.21 mol) of (R)-1-phenylethanol to give 13.1 g (27%) of a diastereomer 2a and 3.0 g (6%) of a diastereomer 2b as white crystals. Their physico-chemical constants were the same as those of the compounds synthesized in the following Example 2.

### EXAMPLE 2

(1) 1-Methylhydantoin (114 g, 1.0 mol) was suspended in 600 mL of 1,2-dichloroethane and heated at 95 to 100° C. on an oil bath. Bromine (57 mL, 1.1 mol) was dropped thereinto with stirring during 6 hours and, after the color of bromine disappeared (within about 1 hour), heating with stirring was still continued for 1 hour more. The solid prepared upon concentration in vacuo was recrystallized from 1,2-dichloroethane and crystals until the third ones were combined to give 166.8 g (86%) of an iminium salt (1-methylhydantoin-5-enium bromide) as white crystals.

(2) Molecular sieve 4A (powdery, 100 g) was suspended in 700 mL of 1,2-dichloroethane and then 25 g (0.2 mol) of (R)-1-phenylethanol and 80 g (0.4 mol) of the above-prepared iminium salt were added thereto. After stirring at 35 to 45° C. for 24 hours, insoluble matters were filtered off and the filtrate was concentrated in vacuo to give a 1:1 mixture of two diastereomers, i.e. (5S,1′R)-1-methyl-5-(1′-phenylethoxy)hydantoin (2a) and (5R,1′R)-1-methyl-5-(1′-phenylethoxy)hydantoin (2b), as an oily substance. When the mixture was separated by flash chromatography and medium-pressure liquid chromatography, crude crystals of the diastereomers 2a and 2b were prepared. Each of the crude crystals was recrystallized from ethyl acetate/hexane to give 15.4 g (33%) of the diastereomer 2a and 17.3 g (37%) of the diastereomer 2b as white crystals.

The Values of Property of (5S,1′R)-1-methyl-5-(1′-phenylethoxy)hydantoin (2a) Mp.: 73-74° C., [α]²⁴D: +159.9° (cl, CHCl₃), Rf: 0.48, ¹H-NMR (CDCl₃): δ 1.54(d, 3H, J=6.5 Hz, CH₃), 2.74(s, 3H, CH₃—N), 4.68(s, 1H, N—CH—C═O), 5.10(q, 1H, J=6.5 Hz, CH7), 7.31-7.41(m, 5H, arom. H), 8.68(s, 1H, NH), IR: νₘₐₓ^{KBr} (cm⁻¹) 3250, 2966, 1794, 1778, 1724, 1705, 1446, 1350, 1315, 1111, 1097, 1066, 756, 694, 559, Elementary analysis (C₁₂H₁₄N₂O₃): calculated value; C 61.53%, H 6.02%, N 11.96%: actual value; C 61.42%, H 6.15%, N 11.86%

The Values of Property of (5R,1′R)-1-methyl-5-(1′-phenylethoxy)hydantoin (2b) Mp.: 106-107.5° C., [α]²⁴D: +148.9° (cl, CHCl₃), Rf: 0.42, ¹H-NMR (CDCl₃): δ 1.55(d, 3H, J=6.5 Hz, CH₃), 2.68(s, 3H, CH₃—N), 4.78(q, 1H, J=6.5 Hz, CH), 5.04(s, 1H, N—CH—C═O), 7.28-7.38(m, 5H, arom. H), 8.07(s, 1H, NH), IR: νₘₐₓ^{KBr} (cm⁻¹) 3265, 2977, 1797, 1776, 1728, 1705, 1444, 1321, 1306, 1095, 1036, 762, 698, 530, Elementary analysis (C₁₂H₁₄N₂O₃): calculated value; C 61.53%, H 6.02%, N 11.96%: actual value; C 61.34%, H 6.09%, N 11.90%

(3) Molecular sieve 4A (powdery, 200 g) was suspended in 1.5 L of 1,2-dichloroethane and then 50 g (0.4 mol) of (S)-1-phenylethanol and 160 g (0.8 mol) of the iminium salt prepared in the above (1) were added thereto. After stirring at 35 to 45° C. for 24 hours, insoluble matters were filtered off and the filtrate was concentrated in vacuo to give 92 g of a 1:1 mixture of two diastereomers, i.e. (5R,1′S)-1-methyl-5-(1′-phenylethoxy)hydantoin (3a) and (5S,1′S)-1-methyl-5-(1′-phenylethoxy)hydantoin (3b), as an oily substance. The mixture was dissolved in 20 mL of ether and cooled (seeds were added thereto if necessary) and the crystals separated out therefrom were filtered to give crude crystals of 20.0 g of a diastereomer 3b. When the residue prepared by concentration of the filtrate was separated by flash chromatography and medium-pressure liquid chromatography, crude crystals of the diastereomers 3a and 3b were prepared. Each of the crude crystals was recrystallized from ethyl acetate/hexane to give 36.4 g (39%) of the diastereomer 3a and 40.3 g (43%) of the diastereomer 3b as white crystals.

The Values of Property of (5R,1′S)-1-methyl-5-(1′-phenylethoxy)hydantoin (3a) Mp.: 73-74° C., [α]²⁴_{D}: −155.2° (cl, CHCl₃), Rf: 0.48, ¹H-NMR(CDCl₃): δ 1.54(d, 3H, J=6.5 Hz, CH₃), 2.74(s, 3H, CH₃—N), 4.68(s, 1H, N—CH—C═O), 5.10(q, 1H, J=6.5 Hz, CH), 7.31-7.41(m, 5H, arom. H), 8.15(s, 1H, NH), IR: νₘₐₓ^{KBr} (cm⁻¹) 3251, 2966, 1794, 1778, 1724, 1705, 1446, 1350, 1315, 1111, 1097, 1066, 756, 694, 559, Elementary analysis (C₁₂H₁₄N₂O₃): calculated value; C 61.53%, H 6.02%, N 11.96%: actual value; C 61.36%, H 6.12%, N 11.94%

The Values of Property of (5S,1′S)-1-methyl-5-(1′-phenylethoxy)hydantoin (3b) Mp.: 101-102° C., [α]²⁴_{D}: −148.7° (cl, CHCl₃), Rf: 0.42, ¹H-NMR (CDCl₃): δ 1.55(d, 3H, J=6.5 Hz, CH₃), 2.67(s, 3H, CH₃—N), 4.77(q, 1H, J=6.5 Hz, CH), 5.04(s, 1H, N—CH—C═O), 7.27-7.37(m, 5H, arom. H), 8.63(s, 1H, NH), IR: νₘₐₓ^{KBr} (cm⁻¹) 3261, 2977, 1797, 1776, 1730, 1705, 1444, 1321, 1306, 1095, 1036, 762, 700, 530, Elementary analysis (C₁₂H₁₄N₂O₃): calculated value; C 61.53%, H 6.02%, N 11.96%: actual value; C 61.45%, H 6.05%, N 12.00%

(4) The compound 2b (23.4 g, 0.1 mol) was dissolved in 250 mL of ethyl acetate, 2.5 g of 10% palladium-carbon was added thereto and the mixture was stirred in a hydrogen gas atmosphere at room temperature for 48 hours. The filtrate after filtering off the 10% palladium carbon was concentrated in vacuo to give crude crystals. They were recrystallized from ethyl acetate to give 9.1 g of (R)-(+)-5-hydroxy-1-methylhydantoin as white crystals. Mp.: 147.5-148.5° C., [α]²⁴_{D}: +8.80 (cl, MeOH), ¹H-NMR (DMSO-d₆): δ 2.74(s, 3H, CH₃), 4.96(d, 1H, J=8.8 Hz, CH), 6.86(d, 1H, J=8.8 Hz, OH), 10.75(s, 1H, NH), IR: νₘₐₓ^{KBr} (cm⁻¹) 3394, 3174, 3076, 2750, 1792, 1741, 1454, 1109, Elementary analysis (C₄H₆N₂O₃): calculated value; C 36.93%, H 4.65%, N 21.53%: actual value; C 37.05%, H 4.66%, N 21.39%

(5) The same treatment was carried out using 42.2 g (0.18 mol) of the compound 3a to give 17.9 g of (R)-(+)-5-hydroxy-1-methylhydantoin as crystals. Mp.: 146.5-149.5° C., [α]²⁴_{D}: +8.90 (cl, MeOH), ¹H-NMR (DMSO-d₆): δ 2.74(s, 3H, CH₃), 4.96(d, 1H, J=8.8 Hz, CH), 6.86(d, 1H, J=8.8 Hz, OH), 10.74(s, 1H, NH), IR: ν_{KBr} ^{(cm}⁻¹) 3396, 3174, 3082, 2750, 1790, 1741, 1452, 1109, Elementary analysis (C₄H₆N₂O₃): calculated value; C 36.93%, H 4.65%, N 21.53%: actual value; C 37.27%, H 4.72%, N 21.43%

(6) The same treatment was carried out using 14.1 g (0.06 mol) of the compound 2a to give 5.7 g of (S)-(−)-5-hydroxy-1-methylhydantoin as crystals. Mp.: 147.0-149.5° C., [α]²⁴_{D}: −9.0° (cl, MeOH), ¹H-NMR (DMSO-d₆): δ 2.74(s, 3H, CH₃), 4.96(d, 1H, J=8.8 Hz, CH), 6.86(d, 1H, J=8.8 Hz, OH), 10.74(s, 1H, NH), IR: νₘₐₓ^{KBr} (cm⁻¹) 3396, 3182, 3076, 2750, 1738, 1454, 1109, Elementary analysis (C₄H₆N₂O₃): calculated value; C 36.93%, H 4.65%, N 21.53%: actual value; C 36.95%, H 4.63%, N 21.37%

(7) The same treatment was carried out using 46.8 g (0.2 mol) of the compound 3b to give 19.3 g of (S)-(−)-5-hydroxy-1-methylhydantoin as crystals. Mp.: 147.0-149.0° C., [α]²⁴_{D}: −8.9° (cl, MeOH), ¹H-NMR(DMSO-d₆): δ 2.74(s, 3H, CH₃), 4.96(d, 1H, J=8.8 Hz, CH), 6.86(d, 1H, J=8.8 Hz, OH), 10.75(s, 1H, NH), IR: νₘₐₓ^{KBr} (cm⁻¹) 3402, 3182, 3076, 2750, 1734, 1450, 1107, Elementary analysis (C₄H₆N₂O₃): calculated value; C 36.93%, H 4.65%, N 21.53%: actual value; C 36.99%. H 4.55%; N 21.59%

### EXAMPLE 3

Optical purity of the optically active substances of 5-hydroxy-1-methylhydantoin prepared above was tested by the following two kinds of methods. The optical purity of the optically active isomer of the present invention is substantially optically pure, for example, over 95% enantiomeric excess or e.e., preferably over 97% e.e., more preferably over 99% e.e.

(1) Proton nuclear magnetic resonance (¹H-NMR) method using a shift reagent

When ¹H-NMR of (±)-5-hydroxy-1-methylhydantoin in heavy acetone was measured together with a shift reagent which is tris[3-(2,2,2-trifluoro-1-hydroxyethylidene)-d-camphorato]europium (III) [Et(tfc)₃] (in 1:6 by weight), each of signals (doublet) of methine group at position 5 were separated into two. In addition, signals (doublet) of hydroxyl group at position 5 were partially overlapped and showed a triplet at a glance. However, ¹H-NMR spectra for (S)-(−)- and (R)-(+)-5-hydroxy-1-methylhydantoin under the same condition showed only a doublet at the corresponding position. Such a fact shows that the above-mentioned (S)-(−)- and (R)-(+)-5-hydroxy-1-methylhydantoin were separated without racemization. Further, from the signal intensity ratio thereof, it was concluded that the optical purity of both isomers (enantiomeric excess or e.e.) was 99.5% e.e. or higher.

(2) High-performance liquid chromatography (HPLC) method

We succeeded in a direct optical resolution of (±)-5-hydroxy-1-methylhydantoin using a chiral column (Chiral Pack AS; Daicel) filled with a stationary phase in which silica gel was coated with amylose to which (S)-1-phenylethyl carbamate was chemically bonded. Thus, when (±)-5-hydroxy-1-methylhydantoin was analyzed using a Chiral Pack AS column and using hexane to which ethanol was added as a mobile phase, a good separation of enantiomer was noted. Further, acetic acid (0.1%) was added to the column and the column temperature was lowered from room temperature to 0° C. to achieve an almost complete separation.

When an HPLC analysis was carried out under the above-mentioned condition, (±)-5-hydroxy-1-methylhydantoin gave two peaks of the area ratio of nearly 1:1 in which retention times were 11.7 minutes and 13.7 minutes. Resolution (Rs) of the two peaks was 2.74 and was higher than Rs=1.5 which is an index for complete separation. Incidentally, (R)-(+)-5-hydroxy-1-methylhydantoin and (S)-(−)-5-hydroxy-1-methylhydantoin prepared by catalytic reduction of the diastereomers 3a and 3b gave only each one peak and no peak for another enantiomer was confirmed. Under the HPLC condition as such, detection of contamination of as small as 0.1% of an optical antipode is possible and, therefore, optical purity of (S)-(−) and (R)-(+)-5-hydroxy-1-methylhydantoin was concluded to be 99.8% e.e. or higher.

### EXAMPLE 4

(1) Pharmacological Action

Adenine was orally administered to Wistar-strain male rats of eight weeks age to prepare a renal insufficiency model. As a result of administration of adenine (200 mg/kg) every day, creatinine level in blood which is an index for renal functions gradually increased in such a manner that 0.44±0.02 mg/dL before administration rose to 4.12±0.53 mg/dL after three weeks. Further, a value of creatinine clearance (mL/kg/hr) decreased from 315.3±13.8 to 25.7±4.4 whereupon renal functions lowered to about one-tenth.

From the 7th to the 17th days from administration of adenine, the test drug was orally administered to the rat in which renal insufficiency was induced by adenine. The creatinine levels in blood before and after the administration of the test drug were compared whereupon a suppressive action of the test drug to an increase of creatinine accompanied by the progress of renal insufficiency was checked. In a control group where no test drug was administered, an increase of creatinine to an extent of 4.37±0.76 mg/dL was noted while, in a group to which (S)-(−)-5-hydroxy-1-methylhydantoin (200 mg/kg/day) was administered, an increase of creatinine was suppressed to an extent of 2.72±0.55 mg/dL. Thus, it was noted that the S-form of the present invention has a suppressive action to renal insufficiency the same as the racemate.

(2) Pharmacokinetics in Blood

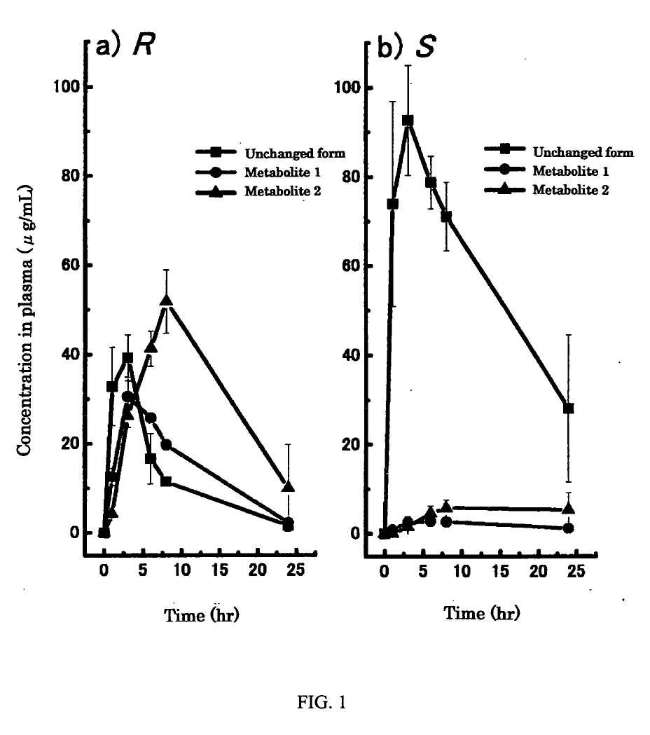

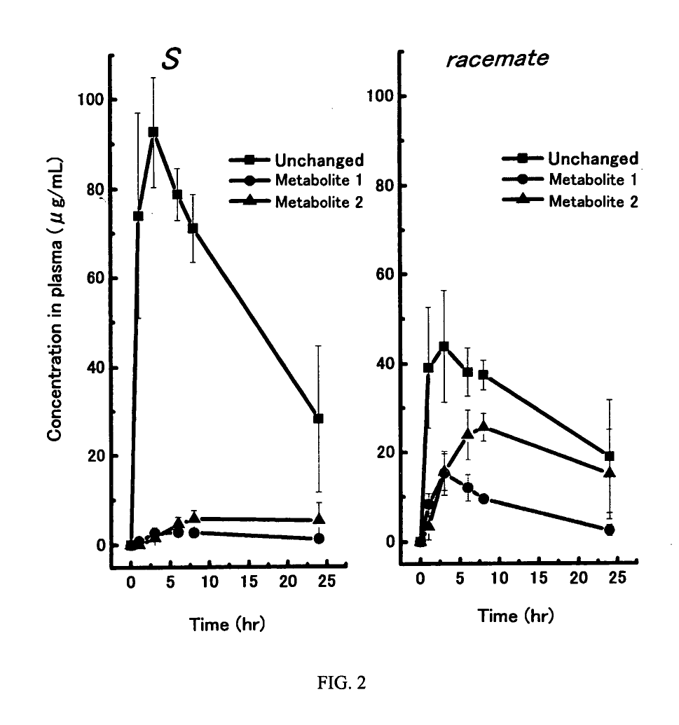

After each 100 mg/kg of (S)-(−)-5-hydroxy-1-methylhydantoin, (R)-(+)-5-hydroxy-1-methylhydantoin, and racemate thereof was orally administered once to the above-mentioned model rats of renal insufficiency (on the 23rd day from administration of adenine), changes in concentration of an unchanged compound and metabolites (metabolite 1: 1-methylimidazolidintrion and metabolite 2: 5-methyloxaluric acid) in plasma were checked. The result is shown in FIG. 1 and FIG. 2. In the renal insufficiency model rats, it was made clear that the S-form of the present invention was rarely converted to its metabolites as compared with the R-form which is an antipode thereof and the racemate.

(3) Toxicity Test

In an acute toxicity test where (±)-5-hydroxy-1-methylhydantoin was once administered intravenously to SD-strain rats (6 weeks age), there was no dead case even when the dose was 2,000 mg/kg, and so the lethal dose of the unchanged compound was presumed to be not less than 2,000 mg/kg. In the same toxicity test however, a lethal dose of the metabolite 1 was about 150 to 300 mg/kg. As such, it is likely that the toxicity of the metabolite 1 is relatively high and, from the result of kinetics of the optical isomer in blood as shown in FIG. 1 and FIG. 2, the S-form is hardly metabolized to the metabolite 1 as compared with the R-form and the racemate whereby it was suggested that the S-form had a higher safety than the R-form and racemate. When each optical isomer was once administered to the mice orally and minimum doses for expression of general symptoms were studied, the R-form showed an expression of symptoms in a dose of one-fifth as compared with the S-form whereby it was noted that the S-form was better in view of adverse actions as well. Furthermore, it was observed that the racemate caused the decrease of general symptoms such as passivity, tactile reaction, pain sensation reaction and ipsilateral flexor reaction at a dose of 200 mg/kg. The same decrease of general symptoms was observed at a dose of 500 mg/kg of the S-form and, consequently, the racemate expressed adverse reactions at lower dose than that of the S-form.

As shown in the above Example 4, and FIG. 1 and FIG. 2, (S)-(−)-5-hydroxy-1-methylhydantoin has better pharmacokinetics in vivo and higher safety as compared with the R-form and the racemate. In renal insufficiency, waste products and toxic substances such as uremic toxin are not excreted but are accumulated in the body due to disturbance of renal functions. Therefore, it is necessary that such accumulation is taken into consideration even for drugs which are administered to patients. Thus, it is preferred for a drug that is to be administered to renal failure patients to be less toxic per se and that the toxicity of metabolites is also low or that the drug hardly changes to metabolites having high toxicity. Since the S-form compound of the present invention rarely changes to the metabolite 1 having high toxicity, it is very useful especially when used as an agent for renal insufficiency.

What is claimed is:

1. (S)-(−)-5-hydroxy-1-methylimidazolidine-2,4-dione or a salt thereof.

2. A composition comprising a substantially optically pure compound according to claim 1 or a salt thereof.

3. A composition according to claim 2, wherein the substantially optically pure (S)-(−)-5-hydroxy-1-methylimidazolidine-2,4-dione or a salt thereof comprises over 95% enantiomeric excess.

4. A method for treating or preventing renal failure, comprising:
administering to a patient in need of such treatment or prevention a pharmaceutically effective amount of the compound according to claim 1 or a pharmaceutically acceptable salt thereof, thereby treating or preventing renal failure.

5. A method according to claim 4, wherein the renal failure comprises acute renal failure.

6. A method according to claim 4, wherein the renal failure comprises chronic renal failure.

7. A method for suppressing the progression of renal failure, comprising:
administering to a patient in need of such treatment a pharmaceutically effective amount of the compound according to claim 1 or a pharmaceutically acceptable salt thereof, thereby suppressing the progression of renal failure.

8. A method according to claim 7, wherein the administering is by oral administration.

9. A method according to claim 8, wherein the effective amount of (S)-(−)-5-hydroxy-1-methylimidazolidine-2,4-dione or a pharmaceutically acceptable salt thereof is administered as a tablet.

10. A method for preparing optical isomers of 5-hydroxy-1-methylhydantoin, comprising:
reacting (±)-5-hydroxy-1-methylhydantoin with (S)-1-phenylethanol or (R)-1-phenylethanol in the presence of an acid catalyst to synthesize a mixture of two diastereomers;
separating the mixture to obtain each of the two diastereomers; and
subjecting each diastereomer to a catalytic reduction in the presence of a catalyst to convert the diastereomer into optically active (S)-(−)-5-hydroxy-1-methylhydantoin or optically active (R)-(+)-5-hydroxy-1-methylhydantoin.

11. A method according to claim 10, comprising reacting (±)-5-hydroxy-1-methylhydantoin with (S)-1-phenylethanol to obtain a mixture of diastereomers and separating the mixture to obtain the following two diastereomers: 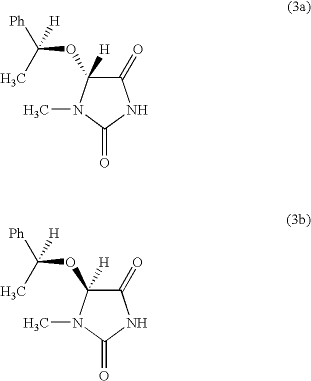
; and
subjecting the diastereomer having formula (3b) to a catalytic reduction in the presence of a catalyst to convert the diastereomer into optically active (S)-(−)-5-hydroxy-1-methylhydantoin.

12. A method according to claim 10, comprising reacting (±)-5-hydroxy-1-methylhydantoin with (R)-1-phenylethanol to obtain a mixture of diastereomers and separating the mixture to obtain the following two diastereomers: 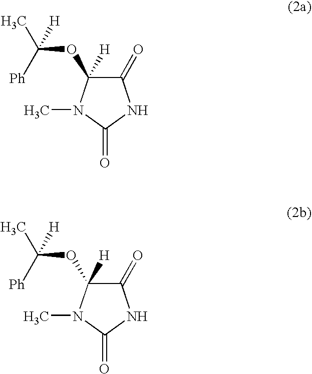
; and
subjecting the diastereomer having formula (2a) to a catalytic reduction in the presence of a catalyst to convert the diastereomer into optically active (S)-(−)-5-hydroxy-1-methylhydantoin.

13. A method according to claim 10, wherein separating the mixture is by column chromatography.

14. A method for preparing optical isomers of 5-hydroxy-1-methylhydantoin, comprising:
reacting 1-methylhydantoin with bromine and recrystallizing to form an iminium salt;
reacting the iminium salt with (S)-1-phenylethanol or (R)-1-phenylethanol to synthesize a mixture of two diastereomers and hydrogen bromide;
removing the hydrogen bromide with an acid scavenger;
separating the mixture to obtain each of the two diastereomers; and
subjecting each separated diastereomer to reduction in the presence of a catalyst to convert the diastereomer into optically active (S)-(−)-5-hydroxy-1-methylhydantoin or optically active (R)-(+)-5-hydroxy-1-methylhydantoin.

15. A method according to claim 14, comprising reacting the iminium salt with (S)-1-phenylethanol to synthesize a mixture of two diastereomers and separating the mixture to obtain the following two diastereomers: 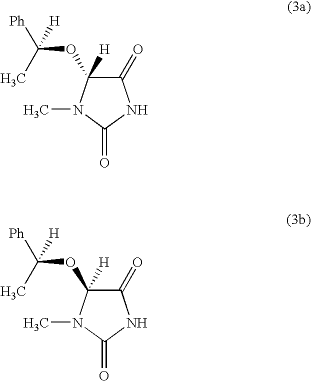
; and
subjecting the diastereomer having formula (3b) to reduction in the presence of a catalyst to convert the diastereomer into optically active (S)-(−)-5-hydroxy-1-methylhydantoin.

16. A method according to claim 14, comprising reacting the iminium salt with (R)-1-phenylethanol to synthesize a mixture of two diastereomers and separating the mixture to obtain the following two diastereomers: 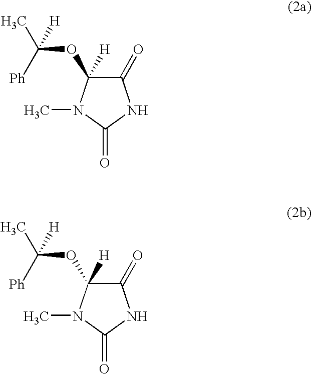
; and
subjecting the diastereomer having formula (2a) to reduction in the presence of a catalyst to convert the diastereomer into optically active (S)-(−)-5-hydroxy-1-methylhydantoin.

17. A method according to claim 14, wherein the acid scavenger is a molecular sieve.

18. A method according to claim 14, wherein the separating comprises at least one of flash chromatography, liquid chromatography, or chiral chromatography.

19. A method for preparing optical isomers of 5-hydroxy-1-methylhydantoin, comprising subjecting (±)-5-hydroxy-1-methylhydantoin to high-performance liquid chromatography using a chiral column to obtain optically active (S)-(−)-5-hydroxy-1-methylhydantoin or optically active (R)-(+)-5-hydroxy-1-methylhydantoin.

20. A method according to claim 19, wherein the chiral column comprises a stationary phase bed of silica gel coated with amylose to which (S)-1-phenylethyl carbamate is chemically bonded.
